(19) United States
(12) Patent Application Publication
(10) Pub. No.: US 2023/0346770 A1
(43) Pub. Date: Nov. 2, 2023

(54) COMBINATION OF BEDAQUILINE, ETHAMBUTOL AND A MACROLIDE IN THE TREATMENT OF NONTUBERCULOUS MYCOBACTERIAL DISEASES

(71) Applicant: Janssen Pharmaceutica NV, Beerse (BE)

(72) Inventors: Ken KUROSAWA, Kanagawa (JP); Sivi MAHADEVAN, Ravels (BE); Chrispin KAMBILI, Maplewood, NJ (US); Tadaishi KOH, Tokyo (JP); Stefaan Louis F. ROSSENU, Lievegem (BE); Wouter Martha M. WILLEMS, Oostakker (BE); Jeike Elise BIEWENGA, Turnhout (BE); Etienne Gilbert Alain ERNAULT, Amsterdam (NE)

(21) Appl. No.: 18/199,748

(22) PCT Filed: Nov. 10, 2021
(86) PCT No.: PCT/EP2021/081193

§ 371 Date: May 19, 2023

Related U.S. Application Data
(60) Provisional application No. 63/113,149, filed on Nov. 12, 2020.

(51) Int. Cl. A61K 31/47; A61K 31/133; A61K 31/7048; A61P 31/04

(52) U.S. Cl. CPC A61K 31/47; A61K 31/133; A61K 31/7048; A61P 31/04

(57) ABSTRACT

The present invention relates to a combination of bedaquiline, a macrolide (e.g. clarithromycin) and, optionally, ethambutol, in a particular treatment regimen, for use in the treatment of a disease associated with nontuberculous mycobacteria (NTM).

### INCORPORATION BY REFERENCE TO ANY PRIORITY APPLICATIONS

This application is a 371 patent application of a national phase entry of International Application No. PCT/EP2021/081193 filed on Nov. 10, 2021, which claims priority from Provisional Application No. 63/113,149 filed on Nov. 12, 2020, the entire contents of which are hereby incorporated by reference in their entirety. Any and all applications for which a foreign or domestic priority claim is identified, for example, in the Application Data Sheet or Request as filed with the present application, are hereby incorporated by reference under 37 CFR 1.57, and Rules 4.18 and 20.6, including PCT Application No. PCT/EP2021/081193 filed on Nov. 10, 2021, and U.S. Provisional Application No. 63/113,149 filed on Nov. 12, 2020.

### FIELD OF THE INVENTION

The present invention relates to a combination of drug components for use in the treatment of a disease associated with nontuberculous mycobacteria, wherein the combination comprises a first drug component that is bedaquiline (such as bedaquiline fumarate, marketed as Sirturo®) and a second drug component that is a macrolide (such as clarithromycin or azithromycin), and optionally a third drug component with activity against nontuberculous mycobacteria (such as ethambutol). The combination may also include an aminoglycoside, such as an aminoglycoside (e.g. injectable or inhalable).

### BACKGROUND OF THE INVENTION

Nontuberculous mycobacterial (NTM) lung disease is a significant cause of morbidity and mortality among individuals with preexisting lung conditions such as bronchiectasis and chronic obstructive pulmonary disease (COPD).

Mycobacterium avium complex (MAC), Mycobacterium abscessus (MAB) and Mycobacterium kansasii are the Mycobacterium species that result in NTM pulmonary disease (NTM-PD). NTM-PD is distinct from the pulmonary infection caused by Mycobacterium tuberculosis. Mycobacterium avium is one of several individual species within the MAC and it accounts for up to 70% of NTM-positive sputum cultures (although there are regional differences). MAC species are naturally-occurring organisms common in water and soil that often colonize in natural water sources such as indoor water systems, hot tubs and pools. MAC-pulmonary disease (MAC-PD) is most often seen in post-menopausal women and patients with underlying lung disease (such as cystic fibrosis or bronchiectasis) or immune deficiencies. Clinical symptoms vary in scope and intensity but commonly include chronic cough, often with purulent sputum, while hemoptysis may also be present. Systemic symptoms include malaise, fatigue, and weight loss in advanced disease.

Current treatment of MAC-PD involves prolonged antibiotic therapy (frequently more than 18 months), with a combination of at least three antibiotics, including a rifamycin (rifampin or rifabutin), a macrolide (azithromycin or clarithromycin), ethambutol and/or aminoglycosides, including injectable or inhalable (amongst others), which are associated with side-effects and a high failure rate. This treatment regimen is currently recommended by the American Thoracic Society (see Griffith et al., Am. J. Respir. Crit. Care Med., 2007, 175, 367-415) and International Guidelines given the in vitro and clinical activity displayed by the combination against MAC. Recently, amikacin liposome inhalation suspension (ALIS, Arikayce®) was approved by the US FDA for the treatment of MAC-PD in adults but otherwise this disease/condition has limited or no alternative treatment options. There are no other antibiotics approved for the treatment of MAC-PD and recommended use of the above agents is merely empirical.

Bedaquiline, or (1R,2S)-1-(6-bromo-2-methoxyquinolin-3-yl)-4-(dimethylamino)-2-naphthalen-1-yl-1-phenylbutan-2-ol, is a Mycobacterium adenosine 5′-triphosphate (ATP) synthase inhibitor that has been developed as a part of a combination therapy for the treatment of pulmonary multidrug-resistant tuberculosis (MDR-TB) in adult patients. Bedaquiline has been approved for that indication under certain conditions under the tradename Sirturo® in territories including the US, Japan, Russia, the EU, South Africa and the Republic of Korea.

The marketed bedaquiline product Sirturo® is a tablet containing bedaquiline fumarate, or (1R,2S)-1-(6-bromo-2-methoxyquinolin-3-yl)-4-(dimethylamino)-2-naphthalen-1-yl-1-phenylbutan-2-ol, fumarate salt, with 100 mg of bedaquiline active ingredient. The fumarate salt can be prepared by reacting the corresponding free base of bedaquiline with fumaric acid in the presence of a suitable solvent, such as for example isopropanol. In the adult population, the first approval in Europe relates to the use of Sirturo® as a part of a combination regimen for pulmonary MDR-TB under certain conditions (when an effective treatment regimen cannot otherwise be composed for reasons of resistance or tolerability). Therein it is indicated (amongst other things) that Sirturo® should be used in combination with at least three other medicinal products to which the patient's isolate has been shown to be susceptible in vitro. If in vitro testing results are unavailable, treatment may be initiated with Sirturo® in combination with at least four medicinal products to which the patient's isolate is likely to be susceptible. The product may also be administered by directly observed therapy (DOT). The recommended dosage is: (i) Weeks 1-2: 400 mg (4 tablets of 100 mg) once daily; and (ii) Weeks 3-24: 200 mg (2 tablets of 100 mg) three times per week (with at least 48 hours between doses). The total duration of treatment with Sirturo® is 24 weeks. Other medicinal products that are used in combination may or should continue after completion of treatment with Sirturo®.

Bedaquiline is known to show activity against Mycobacteria including drug resistant strains, in particular M. tuberculosis, M. bovis, M. avium, M. leprae, M. marinum, M. leprae, M. kansasii, and M. abscessus. The active ingredient, including salt thereof, shows activity against active, sensitive, susceptible Mycobacteria strains and latent, dormant, persistent Mycobacteria strains.

Intl. Pat. Appln. Publ. No. WO 2004/011436 disclosed the activity of the free base of bedaquiline against Mycobacteria. Later documents such as International Patent Application Publication Nos. WO 2005/117875 and WO 2006/067048 disclose the use of bedaquiline in the treatment of inter alia drug resistant tuberculosis and latent tuberculosis. Intl. Pat. Appln. Publ. No. WO 2008/068231 described the suitability of the fumarate salt as a drug product indicating its acceptable bioavailability. The fumarate salt of bedaquiline is described as non-hygroscopic and stable. This document also discloses the preparation of certain formulations and tablets containing bedaquiline fumarate.

Given bedaquiline's in vitro activity against nontuberculous mycobacteria (especially in Mycobacterium abscessus and Mycobacterium avium), there have been reports that it has been used off-label as described by Philley et al., Chest 2015, 148(2), 499-506. This article indicates that bedaquiline has not been tested clinically for efficacy in NTM-mediated disease and describes a small study of patients treated for 1-8 years, already on treatment at the start of bedaquiline therapy, and where 80% had macrolide-resistant isolates. Bedaquiline was administered according to the dosage as had been used in TB trials, and in these studies, the patients were also receiving companion drugs (a mean of 5 drugs total). It is stated in such journal article that “Further study is clearly required to determine whether bedaquiline has a place in the management of NTM lung disease, and if so, to guide its appropriate use.” Intl. Pat. Appln. Publ. No. WO 2020/144197 describes specific drug combinations for the treatment of a disease associated with NTM.

An abstract and certain bedaquiline clinical results were presented at a conference “Advances in the Management of Pulmonary NTM Disease” in San Diego in May 2018, where the topic was entitled “Macrolide Resistant Mycobacterium Avium Complex Lung Disease Treated with Bedaquiline” and it was described that patients (with macrolide resistant MAC lung disease) were administered bedaquiline according to package guidelines in combination with companion drugs given at the discretion of two NTM pulmonary physicians. It was indicated therein that “Treatment options for macrolide resistant MAC lung disease remain limited,” and that “Bedaquiline used with companion therapy may be an option for drug resistant disease . . . ”.

As described herein, there is currently a need for effective treatments for diseases associated with NTM. There is now provided a novel combination for clinical use in the treatment of a disease associated with NTM, as well as a novel method of treatment of a disease associated with NTM.

### BRIEF DESCRIPTION OF THE FIGURES

FIG. 1: Mean concentration-time profile of bedaquiline and M2.

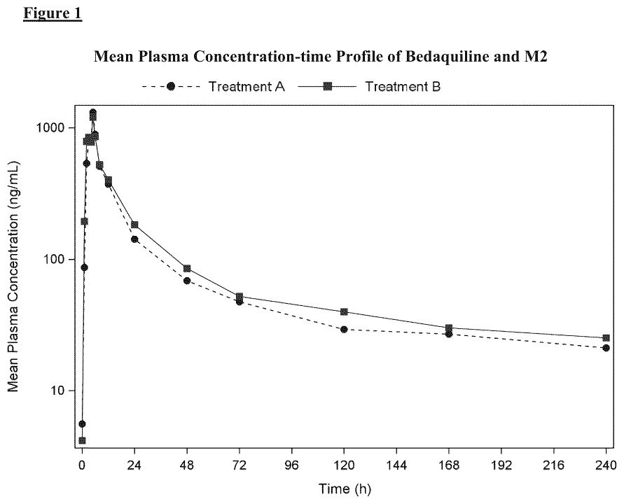

FIG. 2: Mean plasma concentration-time profile of clarithromycin and 14-OH-clarithromycin.

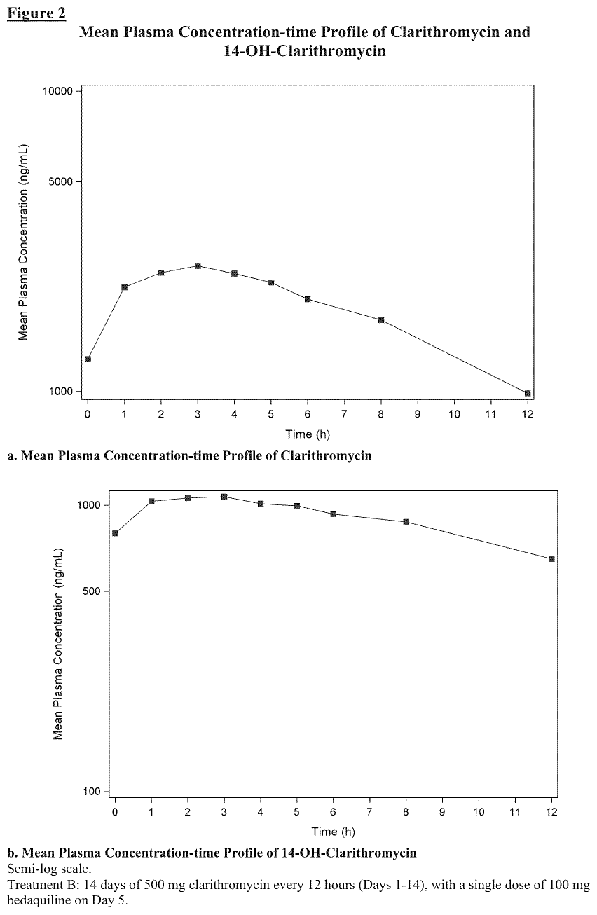

FIG. 3: Simulated Mean Plasma Bedaquiline Trough Concentrations in Standard and Alternative Regimens Based on the Updated Population Pharmacokinetic Model

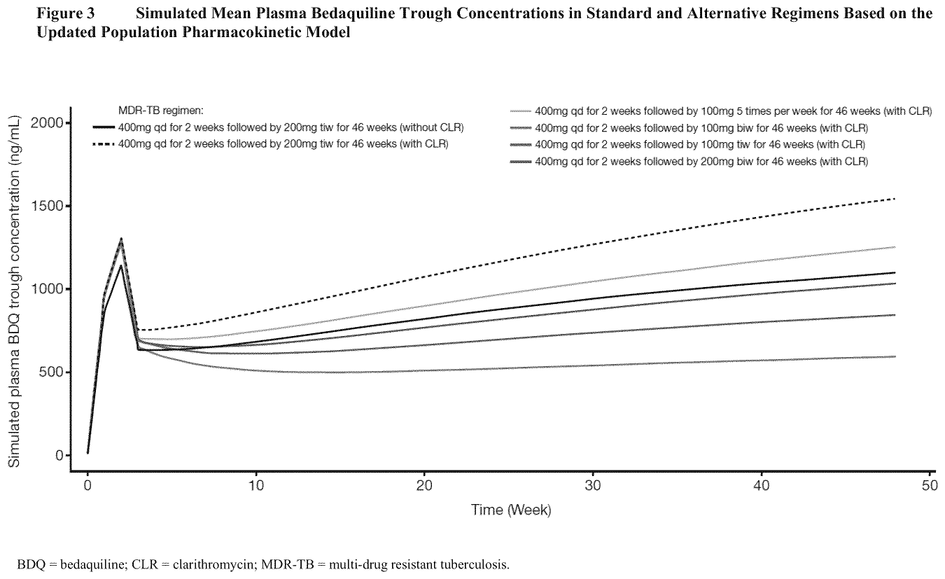

### DESCRIPTION OF THE INVENTION

The present disclosure provides a combination of drug components comprising (e.g. consisting of) a first drug component that is bedaquiline and a second drug component that is a macrolide (e.g. clarithromycin or azithromycin), and, optionally, a third drug component with activity against NTM such as ethambutol, wherein such combination is for use in the treatment of a disease associated with nontuberculous mycobacteria (NTM) in a particular treatment regimen. In an embodiment, the first, second, and optional third drug components are the only drug components in the combination. In an embodiment, the aforementioned combination comprises (e.g. consists of) a first drug component that is bedaquiline and a second drug component that is a macrolide (e.g. clarithromycin or azithromycin). In an embodiment, the first and second drug components are the only drug components in the combination. In an embodiment, the aforementioned combination comprises (e.g. consists of) a first drug component that is bedaquiline, a second component that is a macrolide (e.g., clarithromycin or azithromycin) and a third drug component that is ethambutol. In an embodiment, the bedaquiline is administered in the form of bedaquiline or a salt thereof. In an embodiment, the bedaquiline is administered in the form of bedaquiline fumarate. In an embodiment, the macrolide is clarithromycin. In an embodiment, the clarithromycin is administered in the form of clarithromycin or a salt thereof. In an embodiment, the clarithromycin is administered in the form of clarithromycin. In an embodiment, the macrolide is azithromycin. In an embodiment, the azithromycin is administered in the form of azithromycin or a salt thereof. In an embodiment, the azithromycin is administered in the form of azithromycin dihydrate. In an embodiment, the third drug component is ethambutol. In an embodiment, the ethambutol is administered in the form of ethambutol or a salt thereof, or in the form of ethambutol hydrochloride.

In an embodiment, there is provided a method of treating a disease associated with NTM in a patient in need thereof, comprising administering to the patient an effective amount of a combination of drug components comprising (e.g. consisting of):
(i) a first drug component that is bedaquiline;
(ii) a second drug component that is a macrolide (e.g. clarithromycin or azithromycin); and
(iii) optionally, a third drug component that is ethambutol,

in a particular treatment regimen.

In an embodiment, the combination comprises (e.g. consists of) the first and second drug components. In an embodiment, the first and second drug components are the only drug components in the combination. In an embodiment, the combination comprises (e.g. consists of) the first, second, and third drug components. In an embodiment, the first, second, and third drug components are the only drug components in the combination.

In an embodiment, the bedaquiline is administered in the form of bedaquiline or a salt thereof. In an embodiment, the bedaquiline is administered in the form of bedaquiline fumarate. In an embodiment, the macrolide is clarithromycin. In an embodiment, the clarithromycin is administered in the form of clarithromycin or a salt thereof. In an embodiment, the clarithromycin is administered in the form of clarithromycin. In an embodiment, the macrolide is azithromycin. In an embodiment, the azithromycin is administered in the form of azithromycin or a salt thereof. In an embodiment, the azithromycin is administered in the form of azithromycin dihydrate. In an embodiment, the third drug component is ethambutol. In an embodiment, the ethambutol is administered in the form of ethambutol or a salt thereof, or in the form of ethambutol hydrochloride. In an embodiment, the first, second, and third drug components are the only drug components in the combination.

In embodiment mentioned herein, the combinations of the invention are used in a particular treatment or administration regimen. For instance, the method of treating a disease (associated with NTM) in a patient disclosed herein may have a particular treatment or administration regimen. Such treatment or administration regimen may comprise or consist of the following:
(i) bedaquiline: Weeks 1-2: 400 mg once daily (or “qd”); Weeks 3-24 (and optionally up to 48 weeks, i.e. Weeks 3-48): 200 mg two times per week (e.g. once daily on two different days in a week, e.g., at least 48 hours apart, or at least 72 hours apart);
(ii) the macrolide: for instance, when it is clarithromycin, 800 mg per day, for instance 400 mg twice daily (i.e. 400 mg “bid”) and when it is azithromycin, 250 mg per day;
(iii) ethambutol, if used: 5-50 mg/kg per day (e.g. 1-30 mg/kg per day, such as 15 mg/kg per day); but alternatively, the dosing will be 500-750 mg qd (i.e. daily) or a maximum daily dose of 1 g.

In an embodiment, such a treatment or administration regimen is safe and effective for treating a disease associated with NTM.

These combinations mentioned herein are referred to herein as “the combinations of the invention.” As indicated above, the combinations of the invention may comprise two or three drug components (bedaquiline, a macrolide and, optionally, ethambutol). As used herein, a “drug component” is a medicinal agent that is classified as an antibacterial or antibiotic agent, and/or is active against pathogenic bacteria, such as mycobacteria, and in an embodiment, specifically is active against nontuberculous mycobacteria (especially Mycobacterium avium and Mycobacterium abscessus). In an embodiment, the combinations of the invention contain these two or three drug components as the only drug components. In an embodiment, such combinations also include another drug component (a third or fourth drug component, as appropriate) that is an aminoglycoside (e.g. injectable or inhalable). As used herein, a “macrolide” is an antibiotic or antibacterial macrolide medicinal agent (typically comprising a large, e.g., 14-, 15-, or 16-membered macrocyclic lactone bound to one or more deoxy sugars). Examples of macrolides include erythromycin, clarithromycin, roxithromycin, azithromycin, fidaxomicin, spiramycin, and/or troleandomycin. As used herein, an “aminoglycoside” is an antibacterial medicinal agent with an amino-modified glycoside structure and activity against Gram-negative bacteria. The use of an aminoglycoside may be appropriate for instance in severe cases of the mycobacterial infection or for those patients that do not respond to first-line oral therapy. In an embodiment, and in particular for certain patient populations (for instance, where it is either not needed or can be avoided), an aminoglycoside is not employed. In an embodiment, the aminoglycoside may be any suitable one that has already received approval from a regulatory authority (e.g. for the treatment of Mycobaterium avium complex lung disease, as part of a combination antibacterial regimen), for instance it may be a suitable one that has been approved by the US Food and Drug Administration (FDA) e.g. gentamicin, tobramycin, amikacin, plazomicin, streptomycin, neomycin, and/or paromomycin. In an embodiment, the combinations described herein include bedaquiline, a macrolide, an aminoglycoside, and optionally ethambutol. In an embodiment, the combinations include bedaquiline, a macrolide, an aminoglycoside, and optionally ethambutol as the only drug components in the combination. In such embodiments, the combinations (or the methods of treatment comprising administering such combinations to a patient) do not comprise any other drug components.

It is stated herein that the combinations of the invention may comprise further drug components (in addition to the two requisite drug components and the optional ethambutol), by which we mean that “further drug components” are one (or more) medicinal agent that is classified as an antibacterial or antibiotic agent, by which we include agents that are already known or reported to be an antibacterial or antibiotic and agents that may be tested and achieve a certain antibacterial/antibiotic threshold (e.g. in a standard test or assay, that measures the lowest concentration (in mg/mL) of an antibiotic that inhibits the growth of a give strain, i.e. measuring the MIC value). As such, an antibacterial or antibiotic may be defined as a medicinal agent that achieves an MIC of <3, for instance <1 or <0.5 (although this will depend on the actual test employed). More specifically, where it is indicated herein that a drug has “activity against NTM”, then it may refer to an antibacterial that inhibits the growth of a nontuberculosis mycobacterial strain (for instance, Mycobacterium avium or another known strain) and achieves a certain threshold in a standard test or assay and e.g. MIC (or pIC₅₀/pIC₉₀) values such as those referred to herein. It will be appreciated that antibacterials or antibiotics may act against the bacteria (e.g. mycobacteria) in a bacteriostatic (stopping the bacteria from reproducing but not necessarily killing them) or bacteridical (killing the bacteria) manner. In an embodiment, and as mentioned hereinabove, the combinations of the invention do not comprise any such “further drug components.”

The first, second, and optional third and optional fourth drug components in the combinations of the invention (e.g. bedaquiline, the macrolide and, in an embodiment, ethambutol, and in an embodiment, an aminoglycoside) may be formulated separately (e.g. as defined herein) or one or more of the drug components may be formulated together (e.g. bedaquiline with the macrolide, bedaquiline with ethambutol, macrolide with ethambutol, or bedaquiline with macrolide and ethambutol). In an embodiment, such drug components (for example, bedaquiline, the macrolide and, optionally, ethambutol and/or aminoglycoside) are formulated separately, for instance in the form in which they are marketed/commercially available (for existing approved indications) or in a form as described herein. In an embodiment where such combinations also include another drug component (e.g. third or fourth drug component, as appropriate), for instance an aminoglycoside (such as an injectable or inhalable form), then such components are also formulated separately, e.g. in the form in which they are marketed or as described herein.

In various embodiments (including the method of treating a disease associated with NTM in a patient), the drug components (e.g. bedaquiline, macrolide, optional ethambutol, and optional aminoglycoside) in the combinations of the invention can be co-administered, in other embodiments the drug components of the combinations may be sequentially administered, while in still other embodiments they can be administered substantially simultaneously. In some of the latter embodiments, administration entails taking such drug components (e.g. bedaquiline, macrolide, optional ethambutol, and optional aminoglycoside) within 30 minutes or less of each other, in some embodiments within 15 minutes or less of each other. In some embodiments, the drug components (e.g., bedaquiline, macrolide, optional ethambutol, and optional aminoglycoside) are administered at least once per day (by which we mean, in this instance, the once daily dose, or, one of the twice daily doses i.e. when such drug component is administered bid), at approximately the same time each day. For example, the different drug components are administered within 4 hours of each other on a given administration day, or within 2 hours, or within 1 hour, or in still other embodiments within 30 minutes of each other on a given administration day. However, in an embodiment, the drug components of the combinations of the invention (including bedaquiline, the macrolide and optionally ethambutol and optionally the aminoglycoside) are administered in accordance with existing guidelines (e.g. in accordance with the regulatory label for the indication(s) for which the relevant active is approved).

In some embodiments, the first, second, and optional third drug components of the combinations of the invention are administered as separate oral dosage forms, such as oral capsules or oral tablets. Other formulations may include solid dispersions. In some embodiments, the aminoglycoside is administered as an injection (e.g. subcutaneous or intravenous) and/or as an inhalable (e.g. as a powder, suspension, or liposomal suspension).

The drug components described herein can be used in free drug form or as a pharmaceutically acceptable salt or co-crystal thereof. The drug components (including free forms and salt or co-crystal forms) can be used in solvate forms with suitable solvents, including water (e.g., hydrate form or alcoholate form, in particular a hydrate form such as a hydrate, hemihydrate, monohydrate, or dihydrate). Bedaquiline, clarithromycin, azithromycin, and ethambutol can be used in a free base form or as a suitable pharmaceutically acceptable salt form, such as an acid addition salt form. In an embodiment, bedaquiline is administered in the form of bedaquiline or a salt thereof, such as bedaquiline fumarate. In an embodiment, clarithromycin is administered in the form of clarithromycin. In an embodiment, azithromycin can be used in the form of azithromycin or a salt thereof. Azithromycin includes azithromycin or azithromycin in hydrate form, such as a monohydrate or dihydrate. In an embodiment, azithromycin is administered as azithromycin dihydrate. In an embodiment, ethambutol is administered in the form of ethambutol or a salt thereof, or in the form of ethambutol hydrochloride.

The pharmaceutically acceptable acid addition salts are defined to comprise the therapeutically active non-toxic acid addition salt forms that bedaquiline, azithromycin, clarithromycin, or ethambutol are able to form. Said acid addition salts can be obtained by treating the free form of the drug component with an appropriate acid, for example an inorganic acid, for example hydrohalic acid, in particular hydrochloric acid, hydrobromic acid, sulfuric acid, nitric acid and phosphoric acid; or an organic acid, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicyclic acid, p-aminosalicylic acid and pamoic acid. Conversely, the acid addition salt forms can be converted into the free forms by treatment with an appropriate base. In particular, the fumarate salt is considered for bedaquiline, given that this is the form employed in the already-marketed product Sirturo®. The fumarate salt of bedaquiline can be prepared by reacting the corresponding free base with fumaric acid in the presence of a suitable solvent, such as for example isopropanol. In particular, the hydrochloride salt is considered for ethambutol, as that is the marketed form. In an embodiment, the macrolide (e.g. clarithromycin or erythromycin) and ethambutol can be administered in a form that is commercially available or that is approved for marketing by a health authority.

For the drug components discussed herein, each drug component may be used in a single stereoisomeric form or as a mixture of stereoisomers, if applicable. Whenever reference to bedaquiline is employed herein, we refer to the single stereoisomeric form that is employed in the marketed product Sirturo®, and which is disclosed in Intl. Pat. Appln. Publ. No. WO2004/011436 as an antimycobacterial agent.

For instance, bedaquiline may be administered as a tablet, e.g. formulated as the fumarate salt and containing 100 mg of the active ingredient bedaquiline. When the macrolide employed is clarithromycin, it may be administered as a 500 mg tablet (or, depending on the dose required and the patient, as a suspension, for instance the available suspension containing 250 mg/5 mL). Ethambutol may be administered (depending on the dose required) as ethambutol hydrochloride in a tablet with 100 mg or 400 mg of the ethambutol active ingredient.

For instance, the administration regimens mentioned herein are applicable to a disease associated with NTM as defined/described hereinafter, and in particular relates to NTM-PD. The severity or type of the disease or the severity of the mycobacterial infection may also determine the dose or administration regimen. In an embodiment, the American Thoracic Society (ATS) guidelines may be followed, for guidelines on administration of the macrolide (e.g. clarithromycin) and ethambutol, for the particular disease associated with NTM.

The total treatment regimen may be at least 24 weeks, for instance at least 32 weeks, e.g. about 48 weeks or about 52 weeks (however, in an embodiment, the treatment duration may last up to 18 months or even 24 months). In this respect, the dosing regimen of bedaquiline is already indicated above for a possible 52-week period, and (if the duration runs to 18 or 24 months, then the dosing scheme for the Weeks 3-48 (or the Weeks 3-52 period) will continue); similarly, the dosing of the macrolide (e.g. clarithromycin or azithromycin) and optionally ethambutol will continue for the relevant period e.g. at least 24 weeks, at least 32 weeks, e.g. about 48 weeks or about 52 weeks (or, in a separate embodiment, up to 18 months or up to 24 months). In an embodiment, the treatment regimen also comprises aminoglycosides, for instance an injectable or inhalable form (e.g. in a situation where the ATS guidelines recommend this, for instance when the disease is severe; in this case e.g. a three times weekly injection may be administered). In an embodiment, the treatment regimen does not comprise other drug components; however, companion drugs for instance to treat another disease (for instance which may already be being administered to the patient) may be tolerated (particularly when such drug is for a disease other than a bacterial infection, and e.g. its drug-drug interaction with one or more of the drug components of the combination of the invention has already been studied) although, in an embodiment, any other drugs are not administered during the treatment regimens described herein.

In an embodiment, the macrolide employed in the combinations of the invention is clarithromycin. In an embodiment, the macrolide employed in the combinations of the invention is azithromycin.

In an embodiment, bedaquiline is administered after food (for instance, straight after food or with food), as that may increase the bioavailability of the drug.

In an embodiment, dosing of the macrolide (e.g. clarithromycin or azithromycin) and optionally ethambutol will be as per local guidelines.

In an embodiment, the first, second, and optionally third drug components of the combination of the invention are taken orally, with administration occurring at approximately the same time each day (or, in the case of a drug component is to be administered bid, then such administration refers to one of those twice daily doses).

All dosage amounts mentioned in this disclosure refer to the free base equivalent (i.e. calculated with respect to the free base form of the particular drug component). The values given below represent free-form equivalents, i.e., quantities as if the free form would be administered. If salts are administered the amounts need to be calculated in function of the molecular weight ratio between the salt and the free form.

The daily doses described herein are calculated for an average body weight of about 70 kg and should be recalculated in case of paediatric applications, or when used with patients with a substantially diverting body weight.

It is indicated herein that the combinations used herein are useful in the treatment of a disease associated with nontuberculous mycobacteria (NTM). A method of treatment is also described herein, which relates to treatment of a disease associated with NTM in a patient in need thereof, and the patient is administered an effective amount of a combination of the invention.

As used herein the term “disease associated with NTM in a patient” refers to a patient (or subject, e.g. human patient) being infected with a nontuberculous mycobacteria (especially Mycobacterium abscessus and Mycobacterium avium). In particular, such disease may be a pulmonary disease that is caused by NTM, and thus in an embodiment the disease is NTM-PD. NTM-PD is distinct from the pulmonary infection caused by M. tuberculosis, for which bedaquiline is currently indicated. Mycobacterium avium is one of several species within the MAC and it accounts for up to 70% of NTM-positive sputum cultures (although there are regional differences). MAC species are naturally-occurring organisms common in water and soil that often colonize in natural water sources such as indoor water systems, hot tubs and pools. MAC-PD is most often seen in post-menopausal women and patients with underlying lung disease (such as cystic fibrosis or bronchiectasis) or immune deficiencies. Clinical symptoms vary in scope and intensity but commonly include chronic cough, often with purulent sputum, while hemoptysis may also be present. Systemic symptoms include malaise, fatigue, and weight loss in advanced disease.

Included within the NTM-PD population are treatment-refractory NTM-PD patients, and, as indicated above, the most common NTM-PD is MAC-PD. Hence, in an embodiment, when the term “disease associated with NTM” is referred to herein, this refers to NTM-PD in general, and in a further embodiment, it refers to NTM-PD in treatment-refractory patients; in a further embodiment, it refers to MAC-PD and in a yet further embodiment it refers to MAC-PD in treatment-refractory patients. Treatment-refractory MAC-PD patients are defined as patients who are sputum culture positive for MAC after a minimum of 6 months of guideline-based therapy for MAC-PD infection. Refractory patients treated with the current standard of care have very poor clinical outcomes with approximately 10% ultimately culture converting after 12 months of therapy even with intensification of treatment and the use of aminoglycosides. This patient population therefore also represents an area of unmet medical need. In an embodiment, the disease associated with NTM (e.g. NTM-PD, such as MAC-PD) is accompanied by an underlying lung disease (such as cystic fibrosis, or another as mentioned herein). In an embodiment, the disease associated with NTM is NTM-PD, MAC-PD, fibro-cavitary NTM-PD, or treatment-refractory NTM-PD. In an embodiment, the disease associated with NTM (e.g. NTM-PD, such as MAC-PD) does not include fibro-cavitary NTM-PD. In an embodiment, the disease associated with NTM (e.g. NTM-PD, such as MAC-PD) does not include patients with cystic fibrosis. In an embodiment, a patient in need of treatment as described herein also is diagnosed with bronchiectasis, COPD, asthma, or cystic fibrosis.

Due to its unique mode of action (inhibition of ATP synthase), bedaquiline represents a new class of anti-NTM compounds and currently, no other drugs belonging to the same pharmacological class are available, thus minimizing the potential for cross-resistance. The combinations of the invention described herein thus have an advantage that bedaquiline is a component thereof.

As used herein, “effective amount” refers to the amount of each of the components of the combinations of the invention, or any pharmaceutically acceptable salts thereof, that elicits the biological or medicinal response in a tissue system (e.g., blood, plasma, biopsy) or warm-blooded animal (e.g., human), that is being sought by a health care provider, which includes alleviation of the symptoms of the disease being treated.

Patients treated according to the methods of the disclosure can be “first-line” patients. As used herein, this refers to the patient not having previously received treatment with any drug—investigational or approved—for the disease to be treated (associated with NTM). In a further embodiment, the patients to be treated are not first-line patients, but patients that have already received treatment, for instance patients that have been diagnosed with the disease, still testing positive after 6 months of other guideline therapy (i.e. tested positive in sputum culture for MAC after a minimum of 6 months of guideline-based therapy). Hence, in an embodiment, the patients are treatment-refractory patients or salvage patients. In a further embodiment, the isolates of the NTM are not macrolide-resistant.

To date, the surrogate markers predictive of clinical treatment response have not been defined. The current primary endpoint for “treatment” is sputum culture conversion, defined as 3 consecutive negative monthly sputum cultures by the 6 months timepoint after start of treatment. The primary efficacy outcome time point is selected at 6 months because the majority of the microbiological response occurred during this time-period in a recently completed trial ALIS, for instance as described in Griffith et al., Am. J. Crit. Care Med. 2017, 195(6), 814-823, and Griffith et al., Am. J. Crit. Care Med. 2018, 198(12), 1559-1569.

As mentioned herein, the combination of drug components as described herein may be co-administered, sequentially administered, or administered substantially simultaneously (as described herein). Hence the individual dosage forms of each of the drug components can be administered as separate forms (e.g. as separate tablets or capsules) as described herein.

In an embodiment, there is provided a process for preparing a combination product as defined herein comprising:
bringing into association each of the components (e.g. as separate pharmaceutical formulations) of the combination product and co-packaging (e.g. as a kit of parts) or indicating that the intended use is in combination (with the other components); and/or
bringing into association each of the components in the preparation of a pharmaceutical formulation comprising such components.

In current MAC-PD regimens where a rifamycin is combined with clarithromycin, it is reported that exposure to clarithromycin is suboptimal due to induction of metabolism by the rifamycin component, for instance as described in Shimomura et al., J. Pharm. Health Care Sci. 2015, 1, 32. The combinations of the invention may overcome this. The combinations of the invention may also have the advantage that they are more efficacious, have a better safety profile, and/or have fewer side effects than existing or recommended treatment regimens (e.g. recommended by the ATS). In one embodiment, the combinations of the invention have better efficacy or a lower incidence and/or lower risk of particular side effects, such as death, QT prolongation, nausea, arthralgia, headache, hemoptysis, chest pain, increased levels of serum transaminases, increased levels of blood amylase, anorexia, and rash, than comparative combinations involving administration of bedaquiline three times a week in phase 2 (week 3 forward). In an embodiment, the combinations of the invention have better efficacy or a lower incidence and/or lower risk of particular side effects associated with rifamycin use, including “flu syndrome” (fever, chills, malaise), hematopoietic reactions (leukopenia, thrombocytopenia, or acute hemolytic anemia), shortness of breath, shock, anaphylaxis, and renal failure than a comparative combinations with a rifamycin. In an embodiment, the combinations of the invention produce bedaquiline plasma levels within the safety margins that are already established for bedaquiline, and hence may reduce the incidence of side effects.

The following examples are merely illustrative and are not intended to limit the disclosure to the materials, conditions, or process parameters set forth therein

### EXAMPLES

### Reference Example 1. In Vitro Activity of Bedaquiline

Bedaquiline has a unique spectrum in its specificity to mycobacteria, including atypical species important in humans such as M. avium, M. kansasii, and the fast growers M. fortuitum and M. abscessus. M avium, M. kansasii and M. abscessus can be responsible for causing NTM disease.

Bedaquiline minimum inhibitory concentration (MIC) ranges for M. tuberculosis were ≤0.008 μg/ml to 0.12 μg/ml regardless of resistance sub-type. Bedaquiline MICs were generally <0.1 μg/ml for other mycobacterial species, including species naturally resistant to many other anti-TB agents and involved in opportunistic infections, such as M. avium, M. abscessus. M. fortuitum and M. marinum. In comparison to M. tuberculosis, higher MICs were found for 1 isolate each of M. abscessus (0.25 μg/ml) and M ulcerans (0.50 μg/ml) (see the table below). The activity of bedaquiline appeared to be specific for Mycobacterium species (see Andries et al. Science, 2005, 37, 223-227).

Bedaquiline MIC (μg/ml)

| Mycobacterial Organism | n | MIC range | Median |
|---|---|---|---|
| M. bovis | 1 | — | 0.003 |
| M. avium/M. intracellulare | 7 | 0.007-0.010 | 0.010 |
| (MAC) | | | |
| M. kansasii | 1 | — | 0.003 |
| M. marinum | 1 | — | 0.003 |
| M. fortuitum | 5 | 0.007-0.010 | 0.010 |
| M. abscessus | 1 | — | 0.250 |
| M. smegmatis | 7 | 0.003-0.10 | 0.007 |
| M. ulcerans | 1 | — | 0.500 |

### Example 2: Drug-Drug Interaction Study

Measurements of bedaquiline and its known active metabolite (M2, the methyl-substituted metabolite, formed as a result of N-demethylation of the di-methyl substituted amino group) are referred to herein.

### SUMMARY

Method:

A Phase 1, randomized, crossover study assessed the impact of steady-state clarithromycin (500 mg BID (every 12 hours) for 14 days) on pharmacokinetic (PK) parameters of bedaquiline and its metabolite (M2) after a single-dose bedaquiline (100 mg; n=16).

Overview of Results:

Although no effect was observed on maximum plasma concentration (Cₘₐₓ) of bedaquiline and time to achieve Cₘₐₓ, bedaquiline's mean plasma exposure after a single dose on Day 5 increased by 14% after 10 subsequent days of clarithromycin coadministration, with slower formation of M2.

Simulation Studies

Using these PK data, population pharmacokinetic modeling and simulation were performed to determine the effect of clarithromycin (under steady-state conditions) on bedaquiline exposure. Furthermore, simulations showed that bedaquiline plasma trough concentration was higher (up to 41% until Week 48) when co-administered with clarithromycin (under steady state conditions) as compared to its monotherapy (400 mg once daily [qd] for 2 weeks, followed by 200 mg thrice a week for 46 weeks; referred to herein as the reference regimen). The overall exposure of a simulated bedaquiline regimen (400 mg qd for 2 weeks, followed by 200 mg twice a week [biw] for 46 weeks) with clarithromycin (under steady state conditions) was comparable (<15% difference) to the monotherapy.

### CONCLUSIONS

Based on these results, a combination of bedaquiline (400 mg qd for 2 weeks followed by 200 mg biw for 46 weeks, i.e. twice weekly for 46 weeks) with clarithromycin (under steady state conditions) was selected as a regimen to be explored for efficacy against NTM pulmonary disease (NTM-PD, for instance MAC-PD) and safety.

Advantageously, in the second phase of treatment, the bedaquiline dose could be decreased from three timely weekly to twice weekly based on the results of the drug-drug interaction (and simulation) studies. Such a decrease may ensure that the bedaquiline plasma levels remain within the safety margins that are already established, and hence may avoid potential side effects.

Detailed Methods and Results

Methods

Healthy adults, aged 18 to 55 years (extremes included) with body mass index (BMI) between 18.0 and 30.0 kg/m2 and body weight ≥50 kg at screening were included. This was a Phase 1, single-center, 2-sequence, open-label, randomized, 2-way crossover study conducted in healthy adults to assess the drug-drug interaction between a single-dose bedaquiline under steady-state conditions of clarithromycin.

The subjects were assigned the following treatment groups based on a computer-generated randomization schedule:
Treatment A: A single oral dose of 100 mg bedaquiline (1×100-mg commercial tablet formulation, SIRTURO® [Janssen Pharmaceutica NV, Beerse, Belgium]) on Day 1 morning, taken with a standardized breakfast.
Treatment B: Oral doses of 500 mg clarithromycin every 12 hours (q12 h) from Days 1 to 14 (1×500-mg film-coated commercial tablet formulation, CLARITHROMYCIN SANDOZ® [Sandoz NV, Vilvoorde, Belgium]), taken with a standardized breakfast on Day 1 and on morning of Days 2 and 4 to 8 at the study site (other self-administrations at home could be with or without food), and a single oral dose of 100 mg bedaquiline (1×100-mg commercial tablet, SIRTURO®) on Day 5 morning, taken with a standardized breakfast.

Each subject received both treatments sequentially as Sequence A-B or Sequence B-A, with a washout period of at least 28 days between treatments. The duration of the study for each subject was at least 62 days, excluding the screening period.

Pharmacokinetic Evaluations

For determination of plasma concentrations of bedaquiline and M2, blood samples were taken at 2 hours before dosing of bedaquiline until 240 hours after administration of bedaquiline in Treatments A and B. For determination of plasma concentrations of clarithromycin and 14-OH-clarithromycin blood samples were taken at 30 minutes before dosing of bedaquiline and clarithromycin until 12 hours after the administration (at 1, 2, 3, 4, 5, 6, 8, and 12 hours post-dose) on Day 5 of Treatment B.

Based on individual plasma concentration-time data, the following PK parameters were determined for bedaquiline and M2 on Day 1 of Treatment A and Day 5 of Treatment B: maximum observed analyte concentration (Cₘₐₓ), actual sampling time to reach Cₘₐₓ (tₘₐₓ), area under the analyte concentration-time curve (AUC) from 0 to 72 hours (AUC₇₂ₕ), and AUC from 0 to 240 hours (AUC₂₄₀ₕ). The PK parameters of Cₘₐₓ, tₘₐₓ, minimum observed analyte concentration (Cₘᵢₙ), and AUC from 0 to 12 hours (AUC were calculated for clarithromycin and 14-OH-clarithromycin on Day 5 of Treatment B. The metabolite to parent (M/P, M2/bedaquiline [BDQ]) ratios of the following PK parameters were also determined: C_{max, M2/BDQ}, AUC_{72h, M2/BDQ}, and AUC_{240h, M2/BDQ}.

Bioanalytical methods: Plasma samples were analyzed to determine the concentrations of bedaquiline, M2, clarithromycin, and 14-OH-clarithromycin using a validated liquid chromatography-mass spectrometry/mass spectrometry assay in the sponsor's bioanalytical laboratory (PRA Health Sciences, Netherlands for bedaquiline and PPD, United States for clarithromycin measurements). The quantification ranges were 1 to 2,000 ng/mL for bedaquiline/M2, 20 to 10,000 ng/mL for clarithromycin, and 5 to 2,500 ng/mL for 14-OH-clarithromycin.

Methods for Simulation Studies

Using the concentration data of bedaquiline, population pharmacokinetics (popPK) modeling and simulation were performed. The analysis was conducted based on the previously developed popPK model, which was a 4-compartment model with dual zero-order output, for healthy subjects and tuberculosis patients after bedaquiline monotherapy. The previous model was evaluated for its applicability to the data collected from Treatment A (using a maximum-a-posteriori estimation in NONMEM®; visual predictive check [VPC] and goodness-of-fit [GOF] plots were used for model evaluation). The structural model as well as the co-variate model parameters were then applied to the current data (updated model). The details of the model parameters are listed in the table below (McLeay, S. C., Vis, P., van Heeswijk, R. P., Green, B. Population pharmacokinetics of bedaquiline (TMC207), a novel antituberculosis drug. Antimicrob. Agents. Chemother. 58, 5315-5324 (2014)):

### Population Pharmacokinetic Parameters for the Previously Developed and the Updated Model for Bedaquiline

| | | | Updated |
|---|---|---|---|
| | | | model from |
| | | | the Present |
| | Previously developed | | Study |
| | model | | Estimate |
| Parameter (Units) | Estimate | BSV | (RSE, %) |
| CL/F (L/h) | 2.78 | 50.4 | — |
| Effect CLR on CL/F^{a} | — | — | −0.37 (11) |
| V_{c}/F (L) | 164 | 39.1 | — |
| CLₚ₁/F (L/h) | 11.8 | — | — |
| Vₚ₁/F (L) | 178 | — | — |
| CLₚ₂/F (L/h) | 8.03 | — | — |
| Vₚ₂/F (L) | 3010 | — | — |
| CLₚ₃/F (L/h) | 3.58 | — | — |
| Vₚ₃/F (L) | 7350 | — | — |
| FR1 (%) | 58.5 | 113 | — |
| Day 1 (D 1) (h) | 2.22 | — | — |
| TLAG (h) | 1.48 | — | — |
| Day 2 (D 2) (h) | 1.48 | — | — |
| ALAG1 solution (h) | 0.541 | — | — |
| ALAG1 tablet (h)^{a,b} | 0.917 | — | — |
| Study R207910-CDE102 or | 1.51 | — | — |
| TiDP13-C104 on F^{c} | | | |
| Other studies on F^{a,b} | 2.03 | — | — |
| Increase in CL with Black race (%) | 52.0 | — | — |
| Decrease in V_{c} with female | −15.7 | — | — |
| sex (%)^{a,b} | | | |
| Increase in CL for healthy | 37.5 | — | — |
| volunteers or C202 (%)^{b} | | | |
| Between-subject variability on F | — | 39.6 | — |
| RUV (CV %) | 20.6 | — | — |
| RUV on TiDP13-C208 or | 27.7 | — | — |
| TiDP13-C209 (CV %)^{b,c} | | | |
| Correlation CL/V_{c} | 0.407 | — | — |

BSV = between-subject variability; CL/F = apparent clearance; V_{c}/F = apparent central volume of distribution; CLₚ₁₋₃/F = apparent intercompartmental clearances; Vₚ₁₋₃/F = apparent peripheral volumes of distribution; ALAG1 = absorption lag time; TLAG = additional lag time for absorption for the second pathway; KA = absorption rate constant; D1 = duration of input for the first pathway; D2 = duration of input for the second pathway; FR1 = fraction of dose into the depot compartment; RUV = residual unexplained variability; CV = coefficient of variation.
^{a}Covariates used in the simulations of bedaquiline exposure for non-tuberculous mycobacterial patients (Reference [without effect clarithromycin on CL/F] and Regimen A-D).
^{b}Covariates considered in Bayesian posthoc estimates of present study.
^{c}McLeay S C et al, 2014

The effect of clarithromycin on apparent clearance (CL/F) of bedaquiline was then estimated using combined PK data from Treatments A and B. The first-order conditional estimation method was used for parameter estimation, and the following equation was employed:

CL/F=CLₚₒₚ·(1+θ)^{CLRi}

where CLRᵢ is clarithromycin coadministration status value (no coadministration=0 or coadministration=1), CLₚₒₚ is the population central tendency for CL/F without clarithromycin coadministration, and θ is the change in apparent bedaquiline clearance when clarithromycin was coadministered.

Subsequently, using the updated model, bedaquiline PK profile was simulated to assess the impact of clarithromycin coadministration (under steady-state conditions) on bedaquiline exposure, based on the assumptions of 1,000 non-Black subjects (male:female=1:1) and similar disease status of MDR-TB and NTM. A similar bedaquiline exposure level achieved by the MDR-TB regimen (duration: 24 weeks) was targeted for the NTM treatment (duration: 48 weeks), but with clarithromycin coadministration. The plasma bedaquiline trough concentration (C_{trough}) profile of the standard MDR-TB dose regimen with longer treatment duration (i.e. 400 mg bedaquiline once daily [qd] for 2 weeks followed by 200 mg thrice a week [tiw] for 46 weeks) was simulated with or without clarithromycin coadministration (500 mg q12 h for 48 weeks as a standard NTM treatment). The MDR-TB regimen (without clarithromycin) was chosen as a reference regimen based on the similar minimum inhibitory concentration of bedaquiline for NTM and MDR-TB shown in a previous study (Huitric, E., Verhasselt, P., Andries, K., Hoffner, S. E. In vitro antimycobacterial spectrum of a diarylquinoline ATP synthase inhibitor. Antimicrob. Agents. Chemother. 2007, 51, 4202-4204).

The reference regimen (i.e. bedaquiline 400 mg once daily [qd] for 2 weeks, followed by 200 mg thrice a week for 46 weeks; reference regimen) was compared to 4 regimens of bedaquiline (with clarithromycin): Regimen A (400 mg qd for 2 weeks followed by 200 mg twice a week [biw] for 46 weeks), Regimen B (400 mg qd for 2 weeks followed by 100 mg tiw for 46 weeks), Regimen C (400 mg qd for 2 weeks followed by 100 mg biw for 46 weeks), and Regimen D (400 mg qd for 2 weeks followed by 100 mg 5 times per week for 46 weeks).

The popPK analyses were performed using NONMEM 7.3 (ICON plc, Hanover, MD, USA) using Perl-speaks-NONMEM (PsN, Version 4.2.0). Data management, exploratory analyses, diagnostic graphics, post processing of data, and NONMEM outputs were performed using statistical software R (version 3.4.1).

Results

This study enrolled 16 subjects and was conducted at 1 site in Belgium from March 2019 to June 2019. These 16 subjects (9 females and 7 males) were white, with a median age of 43.0 years (range: 24 to 55 years) and median BMI of 22.44 kg/m2 (range: 18.9 to 29.5 kg/m2). Of all patients, 4 patients missed 1 or more doses of clarithromycin. One of these was reported as a major protocol deviation; the subject missed 3 doses of clarithromycin on Day 13 evening, and Day 14 morning and evening. As no impact on PK analytes was anticipated, no action was taken.

Pharmacokinetics Findings—Bedaquiline and M2

Following a single-dose administration of bedaquiline in Period 1 of Sequence A-B (bedaquiline monotherapy) and Sequence B-A (coadministration with clarithromycin), the pre-dose plasma concentrations of bedaquiline and M2 were quantified in Period 2 in all subjects. Accordingly, the plasma concentrations of bedaquiline and M2 prior to the Period 2 single dose of bedaquiline were <5% and >10% respectively, of the Cₘₐₓ results that were obtained after the single dose of bedaquiline in Period 2.

The bedaquiline mean plasma concentration was maximum at 5 hours post-dose in both treatment groups, after which it rapidly declined initially followed by a slower decline and was quantifiable until 240 hours post-dose. The M2 mean plasma concentration reached a peak at 12 hours post-dose in both treatment groups, after which it decreased slowly (a slight re-increase was noted after Treatment B before the gradual decrease in plasma concentration); M2 was formed slowly but was quantifiable up until 240 hours. Overall, bedaquiline plasma concentrations were slightly higher with lower M2 levels when co-administered with clarithromycin as compared to monotherapy (Error! Reference source not found).

The PK parameters of bedaquiline and M2, and summary of statistical analysis are presented in the table below, respectively. Although mean tₘₐₓ and Cₘₐₓ of bedaquiline were similar in both treatment groups, mean AUCs were slightly higher in Treatment B as compared to Treatment A. Also, higher values were observed in Period 2 compared with Period 1 for both AUC₇₂ₕ (5%-6% higher) and AUC₂₄₀ₕ (8%-18% higher). M2 was formed more slowly in Treatment B (median tₘₐₓ of 23.91 hours for Treatment B vs. 12.00 hours for Treatment A), and Cₘₐₓ and AUCs (including their M/P ratios) were markedly decreased. All M2 PK parameters were higher in Period 2 as compared to Period 1 (36%, 41%, and 46% higher for Cₘₐₓ, AUC₇₂ₕ, and AUC₂₄₀ₕ in Treatment A and 2.2-, 2.1- and 2.1-fold higher in Treatment B, respectively).

Pharmacokinetic Results of Bedaquiline and its Metabolite M2

| Parameters | Treatment A | | Treatment B | |
|---|---|---|---|---|
| (mean [SD]) | Period 1 | Period 2 | Period 1 | Period 2 |
| n | 8 | 8 | 8 | 8 |

Bedaquiline

| | | | | | | | | |
|---|---|---|---|---|---|---|---|---|
| Cₘₐₓ (ng/mL) | 1387 | (407) | 1287 | (432) | 1295 | (343) | 1363 | (351) |
| tₘₐₓ (h)* | 5.00 | (2.00-5.00) | 5.00 | (3.00-5.00) | 5.00 | (2.00-6.03) | 3.48 | (1.98-4.98) |
| AUC_{72 h} | 13886 | (2653) | 14646 | (4469) | 15389 | (4155) | 16357 | (3289) |
| (ng · h/mL) | | | | | | | | |
| AUC_{240 h} | 17641 | (4052) | 20761 | (6341) | 20869 | (5738) | 22555 | (5100) |
| (ng · h/mL) | | | | | | | | |

M2

| | | | | | | | | |
|---|---|---|---|---|---|---|---|---|
| Cₘₐₓ (ng/mL) | 12.0 | (3.27) | 16.3 | (3.83) | 4.66 | (1.54) | 9.89 | (2.04) |
| tₘₐₓ (h)* | 12.00 | (5.00-239.77) | 12.00 | (6.00-72.28) | 35.92 | (11.92-120.20) | 23.90 | (11.92-220.62) |
| AUC_{72 h} | 637 | (177) | 898 | (211) | 260 | (78.1) | 543 | (132) |
| (ng · h/mL) | | | | | | | | |
| AUC_{240 h} | 1839 | (422) | 2691 | (715) | 886 | (268) | 1884 | (370) |
| (ng · h/mL) | | | | | | | | |

M/P Ratios

| | | | | | | | | |
|---|---|---|---|---|---|---|---|---|
| M/P ratio Cₘₐₓ^{#} | 0.00936 | (0.00318) | 0.0142 | (0.00514) | 0.00390 | (0.00153) | 0.00798 | (0.00294) |
| M/P ratio | 0.0481 | (0.0154) | 0.0670 | (0.0190) | 0.0183 | (0.00639) | 0.0347 | (0.00872) |
| AUC_{72 h}^{#} | | | | | | | | |
| M/P ratio | 0.110 | (0.0299) | 0.141 | (0.0390) | 0.0460 | (0.0155) | 0.0881 | (0.0202) |
| AUC_{240 h}^{#} | | | | | | | | |

AUC_{72 h} = area under the analyte concentration-time curve from 0 to 72 hours; AUC_{240 h} = area under the analyte concentration-time curve from 0 to 240 hours; Cₘₐₓ = maximum observed analyte concentration; Cₘᵢₙ = minimum observed analyte concentration; M/P = metabolite/parent ratio; M/P ratio AUC_{72 h} = AUC_{72 h} for M2 divided by AUC_{72 h} for bedaquiline; M/P ratio AUC_{240 h} = AUC_{240 h} for M2 divided by AUC_{240 h} for bedaquiline; M/P ratio Cₘₐₓ = Cₘₐₓ for M2 divided by Cₘₐₓ for bedaquiline; n = number of subjects; SD = standard deviation; tₘₐₓ = actual sampling time to reach the maximum observed analyte concentration.
*tₘₐₓ is presented in median (range).
^{#}M/P ratios corrected for molecular weight (bedaquiline: 555.50 g/mol and M2: 541.47 g/mol). Treatment A: single dose of 100 mg bedaquiline on Day 1; Treatment B: 14 days of 500 mg clarithromycin every 12 hours (Days 1-14), with a single dose of 100 mg bedaquiline on Day 5.

Overall, clarithromycin, when co-administered with bedaquiline, had no impact on tₘₐₓ and Cₘₐₓ of bedaquiline absorption but increased the AUCs (12% for AUC₇₂ₕ [p=0.0011] and 14% for AUC₂₄₀ₕ [p=0.0002]) along with a significant period effect for AUC₂₄₀ₕ (p=0.0005). The plasma exposures of M2 (52%, 51%, and 42% for Cₘₐₓ, AUC₇₂ₕ, and AUC₂₄₀ₕ, respectively) and M/P (52%, 56%, and 49% for Cₘₐₓ, AUC₇₂ₕ, and AUC₂₄₀ₕ) were significantly reduced, along with a significant period effect for all PK parameters (p<0.0001).

Clarithromycin and 14-OH-clarithromycin

The mean plasma concentration-time profiles of clarithromycin and 14-OH-clarithromycin are presented in Error! Reference source not found. On Day 5 morning, the pre-dose mean plasma concentrations of clarithromycin and 14-OH-clarithromycin were 1,281 ng/mL and 797 ng/mL, respectively; the concentrations reached to peak at 3 hours post-dose, followed by a gradual decrease until 12 hours post-dose (i.e. before evening dose), indicating that steady-state was achieved. The mean Cₘₐₓ and Cₘᵢₙ were 2,972 ng/mL and 976 ng/mL for clarithromycin, and 1,152 ng/mL and 636 ng/mL for 14-OH-clarithromycin, respectively. The detailed PK results of clarithromycin and 14-OH-clarithromycin after 5 days of clarithromycin administration are presented in the table below.

Pharmacokinetic Results of Clarithromycin and its Metabolite 14-OH-clarithromycin

| | |
|---|---|
| | Treatment B |
| Parameters | Bedaquiline 100 mg + Clarithromycin at |
| (mean [SD]) | 500 mg Every 12 Hours for 14 days - Day 5) |

Clarithromycin

| n | 16 | |
|---|---|---|
| Cₘₐₓ (ng/mL) | 2972 | (1061) |
| tₘₐₓ (h)* | 3.00 | (1.00-8.03) |
| Cₘᵢₙ (ng/mL) | 976 | (280) |
| AUC_{12 h} (ng · h/mL) | 22866 | (6676) |

14-OH-Clarithromycin

| n | 16 | |
|---|---|---|
| Cₘₐₓ (ng/mL) | 1152 | (326) |
| tₘₐₓ (h)* | 2.00 | (0.00-4.00) |
| Cₘᵢₙ (ng/mL) | 636 | (183) |
| AUC_{12 h} (ng · h/mL) | 10849 | (3050) |

AUC_{12 h} = area under the analyte concentration-time curve from 0 to 12 hours; Cₘₐₓ = maximum observed analyte concentration; Cₘᵢₙ = minimum observed analyte concentration; n = number of subjects; SD = standard deviation; tₘₐₓ = actual sampling time to reach the maximum observed analyte concentration.
*tₘₐₓ is presented in median (range).

Modeling Results

Simulations of Steady-State Bedaquiline Pharmacokinetics

Based on the updated model, the simulated plasma bedaquiline C_{trough} with clarithromycin coadministration in MDR-TB regimen was found to be higher at Week 24 (33%, i.e. 1154±576 ng/mL vs. 869±481 ng/mL) and Week 48 (41%, i.e. 1542±832 ng/mL vs. 1095±661 ng/mL) as compared to its monotherapy.

The reference regimen was then compared with 4 regimens of bedaquiline with clarithromycin. Although Regimens B and C showed lower mean plasma bedaquiline C_{trough} profiles (21% and 46%, respectively), the C_{trough} profiles were comparable for Regimens A and D (<14% difference), relative to the reference regimen (Error! Reference source not found.3). On comparing Regimens A and D for Cₘₐₓ, AUC₂₄ₕ, and AUC₁₆₈ₕ at Weeks 2, 24, and 48 with the reference regimen, their exposure profiles were similar (<15% difference), except for Cₘₐₓ at Week 24 in Regimen D (>20% difference, as shown in the table below:

Simulated Bedaquiline Exposure of MDR-TB Regimen without Clarithromycin, Regimen a and Regimen D

| | MDR-TB Regimen | | | Regimen A | | | Regimen D | | |
|---|---|---|---|---|---|---|---|---|---|
| | Wk 2 | Wk 24 | Wk 48 | Wk 2 | Wk 24 | Wk 48 | Wk 2 | Wk 24 | Wk 48 |
| C_{trough} | | | | | | | | | |
| (ng/mL) | | | | | | | | | |
| Mean | 1125 | 850 | 1069 | 1262 | 794 | 1009 | 1258 | 944 | 1230 |
| (SD) | (523) | (489) | (667) | (567) | (420) | (576) | (529) | (463) | (653) |
| Ratio* | — | — | — | 1.12 | 0.93 | 0.94 | 1.12 | 1.11 | 1.15 |
| Cₘₐₓ | | | | | | | | | |
| (ng/mL) | | | | | | | | | |
| Mean | 3274 | 2013 | 2233 | 3372 | 1919 | 2137 | 3358 | 1596 | 1884 |
| (SD) | (1657) | (1059) | (1212) | (1695) | (1002) | (1138) | (1537) | (740) | (918) |
| Ratio* | — | — | — | 1.03 | 0.95 | 0.96 | 1.03 | 0.79 | 0.84 |
| AUC^{#} | | | | | | | | | |
| (ng · h/mL) | | | | | | | | | |
| Mean | 42652 | 180526 | 217615 | 45900 | 161430 | 197865 | 45780 | 191203 | 239657 |
| (SD) | (19684) | (96964) | (126252) | (20487) | (81712) | (107552) | (19023) | (89638) | (121020) |
| Ratio^{#} | — | — | — | 1.08 | 0.89 | 0.91 | 1.07 | 1.06 | 1.10 |

AUC = area under the analyte concentration-time curve; Cₘₐₓ = maximum observed analyte concentration; C_{trough} = trough concentration; SD = standard deviation; wk = week.
*Mean exposure in Regimen A or D/mean exposure in MDR-TB regimen.
^{#}AUC_{24 h} for Week 2, AUC_{168 h} for Week 24 and Week 48.
MDR-TB regimen: 400 mg bedaquiline once daily for 2 weeks followed by 200 mg thrice a week (without clarithromycin);
Regimen A: 400 mg once daily for 2 weeks followed by 200 mg twice a week for 46 weeks with clarithromycin;
Regimen D: 400 mg once daily for 2 weeks followed by 100 mg 5 times per week for 46 weeks with clarithromycin.

### DISCUSSION

As bedaquiline is now being envisioned for testing against NTM, this was the first human trial to assess the PK interaction between bedaquiline and clarithromycin in order to select a suitable dosing regimen/administration regimen for future testing (e.g. Phase 2/3 clinical trials) and treatments. Bedaquiline is a CYP3A4 substrate and clarithromycin may act as a CYP3A4 inhibitor. This study was therefore needed in order to specify a particular dose of bedaquiline both in the loading phase and the maintenance phase. For instance, if clarithromycin increased the exposure of bedaquiline, this study would be informative for the selection of the bedaquiline dose amount and schedule for both phases of treatment (loading and maintenance phase). In this way, this study envisions a safe and effective dosing regimen/administration regimen for the treatment of a disease associated with NTM, e.g. pulmonary NTM (NTM-PD) such as pulmonary MAC infection (MAC-PD).

Following administration of bedaquiline alone or with clarithromycin in Period 1, plasma concentrations of bedaquiline and M2 were evaluated in Period 2 prior to bedaquiline dosing for all subjects (<5% and >10% of Cₘₐₓ associated with the second single dose for bedaquiline and M2, respectively). The plasma concentrations of bedaquiline and M2 were quantifiable up to 240 hours after bedaquiline dosing in both treatments, and a carry-over effect was noted in Period 2 (bedaquiline: higher AUC₇₂ₕ and AUC₂₄₀ₕ; M2: higher Cₘₐₓ, AUC₇₂ₕ, and AUC₂₄₀ₕ). This was due to the relatively short washout period between both treatment periods compared to the very long terminal elimination half-life of bedaquiline (5.5 months), owing to its cationic amphiphilic characteristics (van Heeswijk, R. P., Dannemann, B., Hoetelmans, R. M. Bedaquiline: a review of human pharmacokinetics and drug-drug interactions. J. Antimicrob. Chemother. 2014, 69, 2310-2318). The results were consistent with the previous randomized trial of an 8-week bedaquiline regimen in MDR-TB patients, where bedaquiline and M2 were quantifiable even after 96 weeks of finishing the treatment, with the mean terminal elimination half-lives of 164 and 159 days, respectively (Diacon, A. H., et al. Randomized pilot trial of eight weeks of bedaquiline (TMC207) treatment for multidrug-resistant tuberculosis: long-term outcome, tolerability, and effect on emergence of drug resistance. Antimicrob. Agents. Chemother. 2012, 56, 3271-3276).^{Error! Reference source not found.} Bedaquiline reached a maximum concentration within 5 hours post-dose, which is consistent with the previous studies where Cₘₐₓ was reached within 4-6 hours of drug administration.

Although Cₘₐₓ and tₘₐₓ of bedaquiline were not impacted by clarithromycin co-administration, the mean plasma concentration of bedaquiline was slightly higher (with decreased Cₘₐₓ and AUCs for M2 and M/P ratio). This increased plasma exposure of bedaquiline along with decreased clearance needed to be considered, in view of clarithromycin potentially acting as a CYP3A4 inhibitor. The mean plasma concentration of clarithromycin (in combination with bedaquiline) reached a peak at 3 hours post-dose, followed by a gradual decrease until 12 hours post-dose, and steady-state was achieved in the study; the PK profile of clarithromycin is already well-established.

In summary, clarithromycin coadministration with single-dose bedaquiline had no impact on Cₘₐₓ and tₘₐₓ of bedaquiline; however, its plasma exposure was increased, with reduced plasma exposure of M2. Overall, single-dose bedaquiline also appears to be safe and well-tolerated, either alone or in combination with clarithromycin; however, this needs to be assessed in long-term trials.

Based on popPK modeling, the effect of clarithromycin on apparent bedaquiline clearance relative to its clearance as monotherapy was estimated to be −37%. Further, simulated C_{trough} of bedaquiline with clarithromycin coadministration was found to be higher at Weeks 24 and 48 as compared to its monotherapy, suggesting that a reduction of the bedaquiline dose, especially in the maintenance period, would be warranted. Based on the similar bedaquiline exposure to that of the currently used regimen in MDR-TB, a combination of bedaquiline (400 mg qd for 2 weeks, followed by 200 mg biw for 46 weeks, Regimen A) and clarithromycin (400 mg bid) was found to be a suitable regimen that could be used in NTM-PD (e.g. MAC-PD) treatment; safety and efficacy of this regimen will be confirmed in a future study.

In view of the above, a combination regimen of bedaquiline (400 mg qd for 2 weeks followed by 200 mg biw for 46 weeks) with clarithromycin was selected for further study for the treatment of NTM-PD (e.g. MAC-PD) disease.

### Example 3: In Vivo Testing

Objectives

Primary Objective

The primary objective is to evaluate the efficacy of bedaquiline at Week 24 compared with a rifamycin (rifabutin or rifampin) when administered as part of a treatment regimen with a macrolide (clarithromycin) and ethambutol in adult patients with treatment-refractory MAC-PD.

Secondary Objectives

The secondary objectives are:
Microbiological assessment in 7H10 or 7H11 agar media: To evaluate the efficacy at Week 24 of BDQ compared with rifamycin when administered as part of a treatment regimen with CAM and EB in adult participants with treatment-refractory MAC-LD.
Clinical assessment: To evaluate the efficacy at Week 24 of BDQ compared with rifamycin when administered as part of a treatment regimen with CAM and EB in adult participants with treatment-refractory MAC-LD.
Microbiological assessment: To evaluate the efficacy at Week 48 of the study intervention in adult participants with treatment-refractory MAC-LD.
Clinical assessment: To evaluate the efficacy of the study intervention in adult participants with treatment-refractory MAC-LD.
Microbiological assessment: To evaluate the efficacy at Week 60 of the study intervention in adult participants with treatment-refractory MAC-LD.
To evaluate the safety and tolerability of the study intervention in adult participants with treatment-refractory MAC-LD.
To evaluate the PK of BDQ (and metabolite M2), and CAM (and metabolite 4-OH-CAM [optional]).

Endpoints

Primary Endpoint

Percentage of participants with sputum culture conversion (defined as 3 consecutive negative monthly sputum cultures taken at least 25 days apart) in MGIT (Mycobacterium growth indicator tube) at the 24-week timepoint after start of investigational treatment.

Secondary Endpoints

The secondary endpoints are:
Percentage of participants with sputum culture conversion (defined as 3 consecutive negative sputum cultures taken at least 25 days apart) in 7H10 or 7H11 agar media at Week 24.
Change from baseline in patient-reported health status on total score of SGRQ at Week 24.
   Percentage of participants with sputum culture conversion (defined as 3 consecutive negative sputum cultures taken at least 25 days apart) in MGIT and 7H10 or 7H11 agar media at Week 48;—Percentage of participants with sputum culture negativity in MGIT and 7H10 or 7H11 agar media, respectively, at each visit after Week 2 per Schedule of Activities;—Time to sputum culture conversion (defined as 3 consecutive negative sputum cultures taken at least 25 days apart) in MGIT up to Week 48;—Time to positivity in MGIT up to Week 48.
   Change from baseline in patient-reported health status on total score of SGRQ at Weeks 48 and 60;—Change from baseline in lung function parameters at Weeks 24, 48, and 60;—Percentage of participants who undergo a change in their MAC-LD treatment regimen by Week 24, by Week 48 (Group A) and by Week 60 (Group B).
Percentage of participants with sputum culture conversion (defined as 3 consecutive negative sputum cultures taken at least 25 days apart) in MGIT and 7H10 or 7H11 agar media at Week 60.
Safety and tolerability based on assessment of AEs, clinical laboratory assessments, 12-lead ECG, vital signs, physical examination, visual examination, and audiology up to Week 60.
PK exposures of BDQ (and metabolite M2 [optional]) at Day 1, Weeks 2, 8, 12, 24, and 48, and CAM (and metabolite 4-OH-CAM [optional]) at Day 1, Weeks 2, 8, 12, and 24.

Study Design

This is a multicenter, randomized, open-label, active-controlled, Phase 2a study to evaluate the efficacy of bedaquiline plus a macrolide (clarithromycin) and ethambutol versus a rifamycin plus a macrolide (clarithromycin) and ethambutol in the treatment of adult patients with treatment-refractory NTM-PD due to MAC.

Adult participants with treatment-refractory NTM-PD due to MAC (defined as patients who are sputum culture positive for MAC after a minimum of 6 months of guideline-based therapy) will be enrolled. In an embodiment, subjects with fibro-cavitary NTM-PD and cystic fibrosis will be excluded.

Participants who meet all the eligibility criteria will be randomized in a 1:1 ratio to receive 1 of the following 2 treatment regimens:
Comparator Group A: Rifamycin*+clarithromycin 400 mg twice daily+ethambutol 500-750 mg qd (once daily) (maximum daily dose of 1 g) [alternatively a dosing of ethambutol according to the guideline dosing of 15 mg/kg per day may be used]* participants may receive rifampicin (450 mg once a day, maximum daily dose 600 mg); rifabutin (300 mg once a day) should be considered if rifampicin is ineffective or cannot be used.
Treatment Group B: Bedaquiline**+clarithromycin 400 mg twice daily+ethambutol 500-750 mg qd (once daily) (or a maximum daily dose of 1 g) [alternatively a dosing of ethambutol according to the guideline dosing of 15 mg/kg per day may be used]** participants will be dosed with bedaquiline as follows:
Weeks 1-2: 400 mg (4 tablets of 100 mg) qd.
Weeks 3-48: 200 mg (2 tablets of 100 mg), twice weekly (with at least 48 hours between doses).

Subjects will be randomly assigned to 1 of two treatment groups based on a computer-generated randomization schedule prepared before the study by or under the supervision of the Sponsor. The randomization will be balanced by using randomly permuted blocks.

All study drugs will be taken orally, drug administration should occur at approximately the same time each day.

The study will consist of a screening period (1 month), baseline visit (Day 1), an open-label treatment period of 12 months (Day 1 to Week 48), and a follow-up period of 3 months (Week 48 to Week 60). The entire study duration for each subject will be 15 months. Participants will return for study visits biweekly in the first 3 months, and at week 16, 20, 24, 32, 40, 48 and 60 thereafter.

All subjects will be followed until 120 weeks post-baseline to collect long-term safety and tolerability, pharmacokinetics, TB treatment outcomes, and anti-mycobacterial information. Subjects who prematurely discontinue from study drug and study procedures will be followed up for survival until 120 weeks post-baseline, unless they withdraw from the study (e.g., withdraw consent/assent). The total study duration (including the treatment and follow-up phases, but excluding the screening phase) will be 120 weeks for each participant. The study is considered completed with the last visit of the last participant participating in the study.

In order to improve bioavailability of bedaquiline, it should be administered with food as this may improve bioavailability approximately 2-fold.

Sample Size Determination

Based on the results of 2 clinical trials of amikacin liposome inhalation suspension (Phase 2 and Phase 3) in an analogous population, it is anticipated that the rifamycin-containing regimen in this study will have a sputum culture conversion rate assumed to be equal to 10% after 24 weeks of treatment.

A sample size of 180 participants (90 in Group A: the bedaquiline containing regimen, 90 in Group B: the rifamycin-containing regimen) will have a power of about 90% to show superiority for a 20% difference in proportion of participants having sputum conversion at Week 24 based on a chi-square test (at the 5% 2-sided significance level) on the intent-to-treat (ITT) population.

Statistical Analyses

The chi square test (at the 5% 2-sided significance level) will be used to compare sputum culture conversion rate in MGIT at Week 24. Participants with missing sputum samples that impact the ability to assess sputum culture conversion (ie, 3 consecutive negative sputum cultures taken at least 25 days apart) will be imputed as nonconvertors in the analysis. Participants requiring a treatment regimen change to individualized regimen will also be considered as nonconvertors. A contaminated sputum culture or failed culture occurring between two negative cultures will be interpreted as “no data,” and will be ignored for the assessment of the 3 consecutive negative cultures.

1-2. (canceled)

3. A method of treating a disease associated with nontuberculous mycobacteria (NTM) in a patient in need thereof, comprising administering to the patient an effective amount of a combination of drug components comprising:
(i) a first drug component that is bedaquiline;
(ii) a second drug component that is a macrolide; and
(iii) a third drug component that is ethambutol, and wherein the administration to the patient consists of a particular regimen comprising:
administration of 400 mg bedaquiline once daily for Weeks 1-2; and 200 mg bedaquiline twice per week for Weeks 3-24 (and optionally up to 48 weeks) (with at least 72 hours between doses); or
administration of 400 mg bedaquiline once daily for Weeks 1-2; and 100 mg bedaquiline five times per week for Weeks 3-24 (and optionally up to 48 weeks).

4. The method of claim 3, wherein the administration to the patient consists of a particular regimen comprising: administration of 400 mg bedaquiline once daily for Weeks 1-2; and 100 mg bedaquiline five times per week for Weeks 3-48 weeks).

5. The method of claim 3, wherein the regimen comprises: administration of the macrolide, wherein the macrolide is clarithromycin or azithromycin, and when the macrolide is clarithromycin, administration is at 800 mg per day, and when the macrolide is azithromycin, administration is at 250 mg per day.

6. The method of claim 5, wherein the regimen comprises administering ethambutol at a dose of 500-750 mg qd or maximum daily dose of 1 g.

7. The method of claim 3, wherein the bedaquiline is administered in a form of bedaquiline fumarate.

8. The method of claim 3, wherein the macrolide is clarithromycin.

9. The method of claim 8, wherein the clarithromycin is administered in a form of clarithromycin or a salt thereof.

10. The method of claim 3, wherein the ethambutol is administered in a form of ethambutol hydrochloride.

11. The method of claim 3, wherein the first, second, and third drug components are the only drug components in the combination.

12. The method of claim 3, wherein the regimen is safe and effective in the treatment of Mycobacterium avium complex (MAC)-pulmonary disease.

13. The method of claim 3, wherein the disease associated with NTM is NTM pulmonary disease (NTM-PD).

14. The method of claim 13, wherein the disease is NTM-PD in which isolates of the NTM are not macrolide-resistant.

15. The method of claim 3, wherein the disease associated with NTM is lung disease.

16. The method of claim 3, wherein the disease associated with NTM is Mycobacterium avium complex (MAC)-pulmonary disease.

17. A method of treating a disease associated with nontuberculous mycobacteria (NTM) in a patient in need thereof, comprising administering to the patient an effective amount of bedaquiline, wherein the bedaquiline is administered in combination with a macrolide and ethambutol, and administered in a particular regimen comprising:
administration of 400 mg bedaquiline once daily for Weeks 1-2; and 200 mg bedaquiline twice per week for Weeks 3-24 (and optionally up to 48 weeks) (with at least 72 hours between doses); or
administration of 400 mg bedaquiline once daily for Weeks 1-2; and 100 mg bedaquiline five times per week for Weeks 3-24 (and optionally up to 48 weeks).

18. The method of claim 17, wherein the bedaquiline is administered in a particular regimen comprising: administering 400 mg of bedaquiline once daily for Weeks 1-2; and administering 200 mg of bedaquiline twice per week for Weeks 3-48 (with at least 72 hours between doses)

19. The method of claim 17, wherein the macrolide is administered in a particular regimen comprising: administering 800 mg of clarithromycin per day.

20. The method of claim 17, wherein the ethambutol is administered in a particular regimen comprising: administering ethambutol hydrochloride at 500-750 mg qd or a maximum daily dose of 1 g.

21. The method of claim 17, wherein the bedaquiline is administered in the form of bedaquiline fumarate.

22-23. (canceled)

24. A combination of drug components suitable for co-administration, sequential administration, or administration substantially simultaneously comprising:
a first drug component that is bedaquiline
a second drug component that is a macrolide, and
a third drug component that is ethambutol,
wherein the combination of drug components is suitable for use in the treatment of a disease associated with nontuberculous mycobacteria (NTM), and
wherein the combination of drug components are suitable for administration in a particular regimen comprising:
(a) administration of 400 mg bedaquiline once daily for Weeks 1-2; and 200 mg bedaquiline twice per week for Weeks 3-24 (and optionally up to 48 weeks) (with at least 72 hours between doses); or
(b) administration of 400 mg bedaquiline once daily for Weeks 1-2; and 100 mg bedaquiline five times per week for Weeks 3-24 (and optionally up to 48 weeks).
